(19) United States
(12) Patent Application Publication
(10) Pub. No.: US 2026/0124294 A1
(43) Pub. Date: May 7, 2026

(54) IMMUNOGENIC COMPOSITIONS AGAINST THE OMICRON VARIANT OF SEVERE ACUTE RESPITORY SYNDROME CORONAVIRUS 2 (SARS-COV-2)

(71) Applicants: Medigen Vaccine Biologics Corporation, Zhubei City (TW); Dynavax Technologies Corporation, Emeryville, CA (US)

(72) Inventors: Charles CHEN, Taipei (TW); Tsun-Yung KUO, I-Lan Country (TW); Chung-Chin WU, I-Lan County (TW); Wei-Hsuan TANG, I-Lan County (TW); Chia-En LIEN, Taipei (TW); Yi-Jiun LIN, Taipei (TW)

(73) Assignees: Medigen Vaccine Biologics Corporation, Zhubei City (TW); Dynavax Technologies Corporation, Emeryville, CA (US)

(21) Appl. No.: 19/118,726

(22) PCT Filed: Oct. 9, 2023
(86) PCT No.: PCT/US2023/076336

§ 371 Date: Apr. 4, 2025

Related U.S. Application Data
(60) Provisional application No. 63/414,042, filed on Oct. 7, 2022.

(51) Int. Cl. A61K 39/215; A61K 39/00; A61P 31/14; C07K 14/005

(52) U.S. Cl. CPC A61K 39/215; A61P 31/14; C07K 14/005; A61K 2039/55505; A61K 2039/55561; A61K 2039/575; C12N 2770/20022; C12N 2770/20034

(57) ABSTRACT

The present invention relates to immunogenic compositions against severe acute respiratory syndrome coronavirus 2 (SARS-CoV-2), especially to immunogenic compositions having recombinant SARS-CoV-2 S proteins derived from Omicron subvariants.

### CROSS-REFERENCE TO RELATED APPLICATIONS

This application claims priority to and the benefit of U.S. Provisional Application No. 63/414,042, filed on Oct. 7, 2022, the disclosure of which is incorporated by reference in its entirety.

### SUBMISSION OF SEQUENCE LISTING AS ASCII TEXT FILE

This application includes an electronically submitted sequence listing in XML format. The XML file contains a sequence listing entitled “Omicron S-2P Sequence Listing.xml” which was created on Oct. 6, 2023 and is 82,878 bytes in size. The sequence listing contained in this XML file is part of the specification and is hereby incorporated by reference herein in its entirety.

### BACKGROUND OF THE INVENTION

### 1. Field of the Invention

The present invention relates to immunogenic compositions against severe acute respiratory syndrome coronavirus 2 (SARS-CoV-2), especially to immunogenic compositions having recombinant SARS-CoV-2 S proteins derived from Omicron subvariants.

### 2. Description of the Prior Art

On 31 Dec. 2019, the World Health Organization (WHO) was alerted to several cases of pneumonia in Wuhan City, Hubei Province of China. The viral pathogen did not match any other known virus and was later officially named “severe acute respiratory syndrome coronavirus 2 (SARS-CoV-2).” The official name of the disease caused by SARS-CoV-2 is coronavirus disease 2019 (COVID-19). Common symptoms of COVID-19 include fever, dry cough, fatigue, tiredness, muscle or body aches, sore throat, diarrhea, conjunctivitis, headache, loss of taste or smell, a rash on skin, and shortness of breath. While the majority of cases result in mild symptoms, some progress to acute respiratory distress syndrome (ARDS), precipitated by cytokine storm, multi-organ failure, septic shock, and blood clots. The first confirmed death from the coronavirus infection occurred on Jan. 9, 2020, and as of Sep. 4, 2023, 770,563.467 confirmed cases of COVID-19, including 6,957,216 deaths, have been reported to the WHO (WHO Coronavirus (COVID-19) Dashboard, https://covid19.who.int). The numbers are still growing fast.

Since the beginning of the COVID-19 pandemic, mutants have been detected periodically. A number of them were found to carry mutations in the crucial receptor-binding domain (RBD), a prime target for antibody recognition and neutralization. Among these variants, the newly emerged Omicron (B.1.1.529) variant has 32 changes in its spike protein relative to that of the original virus (Wuhan-hu-1), particularly in RBD and the N-terminal domain, the primary targets of neutralizing antibodies. In just a few weeks since the discovery of Omicron variant, it has turned out to be highly transmissible and less susceptible to vaccines than other variants. The Omicron variant and its subvariants have spread fast around the world and been identified in more than 110 countries. Therefore, vaccines that are effective against the Omicron variant of SARS-CoV-2 are urgently needed.

### SUMMARY OF THE INVENTION

In one aspect, the present invention provides immunogenic compositions against a severe acute respiratory syndrome coronavirus 2 (SARS-CoV-2), comprising an antigenic recombinant protein and an adjuvant selected from the group consisting of an aluminum-containing adjuvant, an unmethylated cytosine-phosphate-guanosine (CpG) motif, and a combination thereof, wherein the antigenic recombinant protein comprises a recombinant protein derived from the spike protein of SARS-CoV-2 Omicron subvariants.

In some embodiments, the immunogenic composition described herein provides one or more of improved immunogenicity, an enhanced immune response, and/or broad-spectrum immunity.

In other embodiments, methods, formulations, articles, devices, and/or preparations for administering the immunogenic composition described herein, which provides improved immunogenicity, an enhanced immune response, and/or a broad-spectrum immunity, to a subject are also disclosed.

Those skilled in the art will recognize, or be able to ascertain using no more than routine experimentation, many equivalents to the specific embodiments of the invention described herein. Such equivalents are intended to be encompassed by the following embodiments.

Embodiment 1. An immunogenic composition against a severe acute respiratory syndrome coronavirus 2 (SARS-CoV-2), comprising an antigenic recombinant protein and an adjuvant selected from the group consisting of an aluminum-containing adjuvant, an unmethylated cytosine-phosphate-guanosine (CpG) motif, and a combination thereof, wherein the antigenic recombinant protein substantially consisting of an S1 subunit and an S2 subunit without a transmembrane domain (TM) and a cytoplasmic tail (CT) of spike protein of a SARS-CoV-2 Omicron subvariant at a N-terminus, with a “GSAS” substitution at an S1/S2 cleavage site (S1/S2) and two proline substitutions at a junction between a heptad repeat 1 (HR1) and a central helix (CH), and a C-terminal T4 fibritin trimerization motif of SEQ ID NO: 2, with or without a cleaved HRV3C protease cleavage sequence of SEQ ID NO: 19 at a C-terminus, and wherein the spike protein of a SARS-CoV-2 Omicron subvariant comprises at least the following common mutations compared to spike protein of SARS-CoV-2 Wuhan strain (SEQ ID NO: 42): G142D, S373P, S375F, K417N, N440K, S477N, T478K, E484A, Q498R, N501Y, Y505H, D614G, H655Y, N679K, P681H, N764K, D796Y, Q954H, and N969K.

Embodiment 2. The immunogenic composition of Embodiment 1, wherein the antigenic recombinant protein is at least 95%, 96%, 97%, 98%, 99%, or 100% identical to SEQ ID NO: 44 or 45.

Embodiment 3. The immunogenic composition of Embodiment 1 or 2, wherein the antigenic recombinant protein is at least one selected from the group consisting of
an S-2P Omicron BA.1 recombinant protein comprising an amino acid sequence at least 98%, 99%, or 100% identical to SEQ ID NO: 14 or 15;
an S-2P Omicron BA.4/BA.5 recombinant protein comprising an amino acid sequence at least 98%, 99%, or 100% identical to SEQ ID NO: 17 or 18;
an S-2P Omicron BA.2.75 recombinant protein comprising an amino acid sequence at least 98%, 99%, or 100% identical to SEQ ID NO: 20 or 21:
an S-2P Omicron BA.2 recombinant protein comprising an amino acid sequence at least 98%, 99%, or 100% identical to SEQ ID NO: 22 or 23;
an S-2P Omicron BA.4.6 recombinant protein comprising an amino acid sequence at least 98%, 99%, or 100% identical to SEQ ID NO: 24 or 25;
an S-2P Omicron BF.7 recombinant protein comprising an amino acid sequence at least 98%, 99%, or 100% identical to SEQ ID NO: 26 or 27;
an S-2P Omicron BQ.1 recombinant protein comprising an amino acid sequence at least 98%, 99%, or 100% identical to SEQ ID NO: 28 or 29;
an S-2P Omicron BQ.1.1 recombinant protein comprising an amino acid sequence at least 98%, 99%, or 100% identical to SEQ ID NO: 30 or 31;
an S-2P Omicron XBB.1.5 recombinant protein comprising an amino acid sequence at least 98%, 99%, or 100% identical to SEQ ID NO: 32 or 33:
an S-2P Omicron XBB.1 recombinant protein comprising an amino acid sequence at least 98%, 99%, or 100% identical to SEQ ID NO: 34 or 35;
an S-2P Omicron XBB.1.16 recombinant protein comprising an amino acid sequence at least 98%, 99%, or 100% identical to SEQ ID NO: 36 or 37;
an S-2P Omicron BA.2.86 recombinant protein comprising an amino acid sequence at least 98%, 99%, or 100% identical to SEQ ID NO: 38 or 39; and
an S-2P Omicron EG.5.1 recombinant protein comprising an amino acid sequence at least 98%, 99%, or 100% identical to SEQ ID NO: 40 or 41.

Embodiment 4. The immunogenic composition of any one of Embodiment 1 to 3, further comprising an S-2P W recombinant protein, substantially consisting of residues 14-1208 of spike protein of SARS-CoV-2 Wuhan strain with proline substitutions at residues 986 and 987 and a “GSAS” substitution at residues 682-685 and a C-terminal T4 fibritin trimerization motif of SEQ ID NO: 2, with or without a cleaved HRV3C protease cleavage sequence of SEQ ID NO: 19 at a C-terminus.

Embodiment 5. The immunogenic composition of Embodiment 4, wherein the S-2P W recombinant protein comprises an amino acid sequence at least 98%, 99%, or 100% identical to SEQ ID NO: 5 or 6.

Embodiment 6. The immunogenic composition of any one of Embodiments 1 to 5, wherein the aluminum-containing adjuvant comprises aluminum hydroxide, aluminum oxyhydroxide, aluminum hydroxide gel, aluminum phosphate, aluminum phosphate gel, aluminum hydroxyphosphate, aluminum hydroxyphosphate sulfate, amorphous aluminum hydroxyphosphate sulfate, potassium aluminum sulfate, aluminum monostearate or a combination thereof.

Embodiment 7. The immunogenic composition of Embodiment 6, wherein a 0.5 ml dose of the immunogenic composition comprises from about 250 to about 1500 μg Al³⁺, or about 375 μg Al³⁺ or about 750 μg Al³⁺.

Embodiment 8. The immunogenic composition of any one of Embodiments 1 to 7, wherein the unmethylated CpG motif comprises a synthetic oligodeoxynucleotide (ODN) of SEQ ID NO: 7, SEQ ID NO: 8, SEQ ID NO: 9, SEQ ID NO: 10, SEQ ID NO: 11, SEQ ID NO: 12, or a combination thereof.

Embodiment 9. The immunogenic composition of Embodiment 8, wherein a 0.5 ml dose of the immunogenic composition comprises from about 750 to about 3000 μg of the synthetic oligodeoxynucleotide, or about 750 μg, 1500 μg, or 3000 μg of the synthetic oligodeoxynucleotide.

Embodiment 10. A method for eliciting an immune response against a severe acute respiratory syndrome coronavirus 2 (SARS-CoV-2) in a subject in need thereof, comprising administering to the subject at least one dose of the immunogenic composition of any one of Embodiments 1 to 9.

Embodiment 11. A method for protecting a subject in need thereof from infection with a severe acute respiratory syndrome coronavirus 2 (SARS-CoV-2), comprising administering to the subject at least one dose of the immunogenic composition of any one of Embodiments 1 to 9.

Embodiment 12. The method of Embodiment 10 or 11, wherein the severe acute respiratory syndrome coronavirus 2 (SARS-CoV-2) is the Omicron variant.

Embodiment 13. A method for preventing a subject in need thereof from contracting COVID-19 disease, comprising administering to the subject at least one dose of the immunogenic composition of any one of Embodiments 1 to 9.

Embodiment 14. The method of Embodiment 13, wherein the COVID-19 disease is caused by SARS-CoV-2 Omicron variant.

Embodiment 15. The method of any one of Embodiments 10 to 14, wherein the subject was administered with at least one dose of an immunogenic composition derived from SARS-CoV-2 Wuhan strain before the administration with the at least one dose of the immunogenic composition of any one of Embodiments 1 to 9.

Embodiment 16. The method of Embodiment 15, wherein the immunogenic composition derived from SARS-CoV-2 Wuhan strain comprises a polynucleotide encoding at least a fragment of spike protein of SARS-CoV-2 Wuhan strain or a polypeptide of at least a fragment of spike protein of SARS-CoV-2 Wuhan strain.

Embodiment 17. The method of Embodiment 16, wherein the at least a fragment of spike protein of SARS-CoV-2 Wuhan strain comprises a polypeptide sequence at least 98%, 99%, or 100% identical to SEQ ID NO: 5 or 6.

Embodiment 18. The method of any one of Embodiments 15 to 17, wherein the immunogenic composition derived from SARS-CoV-2 Wuhan strain comprises a polypeptide sequence at least 98%, 99%, or 100% identical to SEQ ID NO: 5 or 6, and an adjuvant selected from the group consisting of an aluminum-containing adjuvant, an unmethylated cytosine-phosphate-guanosine (CpG) motif, and a combination thereof.

Embodiment 19. The method of Embodiment 18, wherein the aluminum-containing adjuvant comprises aluminum hydroxide, aluminum oxyhydroxide, aluminum hydroxide gel, aluminum phosphate, aluminum phosphate gel, aluminum hydroxyphosphate, aluminum hydroxyphosphate sulfate, amorphous aluminum hydroxyphosphate sulfate, potassium aluminum sulfate, aluminum monostearate or a combination thereof.

Embodiment 20. The method of Embodiment 18 or 19, wherein a 0.5 ml dose of the immunogenic composition comprises from about 250 to about 1500 μg Al³⁺, or about 375 μg Al³⁺ or about 750 μg Al³⁺.

Embodiment 21. The method of any one of Embodiments 18 to 20, wherein the unmethylated CpG motif comprises a synthetic oligodeoxynucleotide (ODN) of SEQ ID NO: 7. SEQ ID NO: 8, SEQ ID NO: 9, SEQ ID NO: 10, SEQ ID NO: 11, SEQ ID NO: 12, or a combination thereof.

Embodiment 22. The method of any one of Embodiments 18 to 21, wherein a 0.5 ml dose of the immunogenic composition comprises from about 750 to about 3000 μg of the synthetic oligodeoxynucleotide, or about 750 μg, 1500 μg, or 3000 μg of the synthetic oligodeoxynucleotide.

Embodiment 23. The method of any one of Embodiments 10 to 22, wherein the immunogenic composition is administered by intramuscular injection.

Embodiment 24. The immunogenic composition of any one of Embodiments 1-9 for use in eliciting an immune response against a severe acute respiratory syndrome coronavirus 2 (SARS-CoV-2) in a subject in need thereof.

Embodiment 25. The immunogenic composition of any one of Embodiments 1-9 for use in protecting a subject in need thereof from infection with severe acute respiratory syndrome coronavirus 2 (SARS-CoV-2).

Embodiment 26. The immunogenic composition of any one of Embodiments 1-9 for use according to Embodiment 24 or 25, wherein the severe acute respiratory syndrome coronavirus 2 (SARS-CoV-2) is the Omicron variant.

Embodiment 27. The immunogenic composition of any one of Embodiments 1-9 for use in preventing a subject in need thereof from contracting COVID-19 disease.

Embodiment 28. The immunogenic composition of any one of Embodiments 1-9 for use according to Embodiment 27, wherein the COVID-19 disease is caused by SARS-CoV-2 Omicron variant.

Embodiment 29. The immunogenic composition of any one of Embodiments 1-9 for use according to any one of Embodiments 24 to 28, wherein the subject was administered with at least one dose of an immunogenic composition derived from SARS-CoV-2 Wuhan strain before the administration with at least one dose of the immunogenic composition of any one of Embodiments 1-9.

Embodiment 30. The immunogenic composition of any one of Embodiments 1-9 for use according to Embodiment 29, wherein the immunogenic composition derived from SARS-CoV-2 Wuhan strain comprises a polynucleotide encoding at least a fragment of spike protein of SARS-CoV-2 Wuhan strain or a polypeptide of at least a fragment of spike protein of SARS-CoV-2 Wuhan strain.

Embodiment 31. The immunogenic composition of any one of Embodiments 1-9 for use according to Embodiment 30, wherein the at least a fragment of spike protein of SARS-CoV-2 Wuhan strain comprises a polypeptide sequence at least 98%, 99%, or 100% identical to SEQ ID NO: 5 or 6.

Embodiment 32. The immunogenic composition of any one of Embodiments 1-9 for use according to and one of Embodiments 29 to 31, wherein the immunogenic composition derived from SARS-CoV-2 Wuhan strain comprises a polypeptide sequence at least 98%, 99%, or 100% identical to SEQ ID NO: 5 or 6, and an adjuvant selected from the group consisting of an aluminum-containing adjuvant, an unmethylated cytosine-phosphate-guanosine (CpG) motif, and a combination thereof.

Embodiment 33. The immunogenic composition of any one of Embodiments 1-9 for use according to Embodiment 32, wherein the aluminum-containing adjuvant comprises aluminum hydroxide, aluminum oxyhydroxide, aluminum hydroxide gel, aluminum phosphate, aluminum phosphate gel, aluminum hydroxyphosphate, aluminum hydroxyphosphate sulfate, amorphous aluminum hydroxyphosphate sulfate, potassium aluminum sulfate, aluminum monostearate or a combination thereof.

Embodiment 34. The immunogenic composition of any one of Embodiments 1-9 for use according to Embodiment 32 or 33, wherein a 0.5 ml dose of the immunogenic composition comprises from about 250 to about 1500 μg Al³⁺, or about 375 μg Al³⁺ or about 750 μg Al³⁺.

Embodiment 35. The immunogenic composition of any one of Embodiments 1-9 for use according to any one of Embodiments 32 to 34, wherein the unmethylated CpG motif comprises a synthetic oligodeoxynucleotide (ODN) of SEQ ID NO: 7, SEQ ID NO: 8, SEQ ID NO: 9, SEQ ID NO: 10, SEQ ID NO: 11, SEQ ID NO: 12, or a combination thereof.

Embodiment 36. The immunogenic composition of any one of Embodiments 1-9 for use according to any one of Embodiments 32 to 35, wherein a 0.5 ml dose of the immunogenic composition comprises from about 750 to about 3000 μg of the synthetic oligodeoxynucleotide, or about 750 μg, 1500 μg, or 3000 μg of the synthetic oligodeoxynucleotide.

Embodiment 37. The immunogenic composition of any one of Embodiments 1-9 for use according to any one of Embodiments 24 to 36, wherein the immunogenic composition is administered by intramuscular injection.

Embodiment 38. Use of the immunogenic composition of any one of Embodiments 1-9 for the manufacture of a medicament for eliciting an immune response against a severe acute respiratory syndrome coronavirus 2 (SARS-CoV-2) in a subject in need thereof.

Embodiment 39. Use of the immunogenic composition of any one of Embodiments 1-9 for the manufacture of a medicament for protecting a subject in need thereof from infection with severe acute respiratory syndrome coronavirus 2 (SARS-CoV-2).

Embodiment 40. The use according to Embodiment 38 or 39, wherein the severe acute respiratory syndrome coronavirus 2 (SARS-CoV-2) is the Omicron variant.

Embodiment 41. Use of the immunogenic composition of any one of Embodiments 1-9 for the manufacture of a medicament for preventing a subject in need thereof from contracting COVID-19 disease.

Embodiment 42. The use according to Embodiment 41, wherein the COVID-19 disease is caused by SARS-CoV-2 Omicron variant.

Embodiment 43. The use according to any one of Embodiments 38 to 42, wherein the subject was administered with at least one dose of an immunogenic composition derived from SARS-CoV-2 Wuhan strain before the administration with at least one dose of the immunogenic composition of any one of Embodiments 1-9.

Embodiment 44. The use according to Embodiment 43, wherein the immunogenic composition derived from SARS-CoV-2 Wuhan strain comprises a polynucleotide encoding at least a fragment of spike protein of SARS-CoV-2 Wuhan strain or a polypeptide of at least a fragment of spike protein of SARS-CoV-2 Wuhan strain.

Embodiment 45. The use according to Embodiment 44, wherein the at least a fragment of spike protein of SARS-CoV-2 Wuhan strain comprises a polypeptide sequence at least 98%, 99%, or 100% identical to SEQ ID NO: 5 or 6.

Embodiment 46. The use according to any one of Embodiments 43 to 45, wherein the immunogenic composition derived from SARS-CoV-2 Wuhan strain comprises a polypeptide sequence at least 98%, 99%, or 100% identical to SEQ ID NO: 5 or 6, and an adjuvant selected from the group consisting of an aluminum-containing adjuvant, an unmethylated cytosine-phosphate-guanosine (CpG) motif, and a combination thereof.

Embodiment 47. The use according to Embodiment 46, wherein the aluminum-containing adjuvant comprises aluminum hydroxide, aluminum oxyhydroxide, aluminum hydroxide gel, aluminum phosphate, aluminum phosphate gel, aluminum hydroxyphosphate, aluminum hydroxyphosphate sulfate, amorphous aluminum hydroxyphosphate sulfate, potassium aluminum sulfate, aluminum monostearate or a combination thereof.

Embodiment 48. The use according to Embodiment 46 or 47, wherein a 0.5 ml dose of the immunogenic composition comprises from about 250 to about 1500 μg Al³⁺, or about 375 μg Al³⁺ or about 750 μg Al³⁺.

Embodiment 49. The use according to any one of Embodiments 46 to 48, wherein the unmethylated CpG motif comprises a synthetic oligodeoxynucleotide (ODN) of SEQ ID NO: 7, SEQ ID NO: 8, SEQ ID NO: 9, SEQ ID NO: 10. SEQ ID NO: 11, SEQ ID NO: 12, or a combination thereof.

Embodiment 50. The use according to any one of Embodiments 46 to 49, wherein a 0.5 ml dose of the immunogenic composition comprises from about 750 to about 3000 μg of the synthetic oligodeoxynucleotide, or about 750 μg, 1500 μg, or 3000 μg of the synthetic oligodeoxynucleotide.

Embodiment 51. The use according to any one of Embodiments 38 to 50, wherein the immunogenic composition is administered by intramuscular injection.

These and other aspects will become apparent from the following description of the preferred embodiment taken in conjunction with the following drawings.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment.

FIGS. 1A and 1B show a summary of pseudovirus-based neutralization assays in BALB/c mice 2 weeks after the third immunization. BALB/c mice (N=5 per group) were immunized twice at 3 weeks apart with two doses of S-2P W recombinant protein followed by one dose of S-2P W recombinant protein (Group A, W+W+W), one dose of S-2P Omicron BA.1 recombinant protein (Group B, W+W+BA.1), one dose of S-2P Omicron BA.4/BA.5 recombinant protein (Group C, W+W+BA.4/BA.5), one dose of the bivalent mixture of S-2P W and S-2P Omicron BA.1 recombinant proteins (group D), or one dose of the bivalent mixture of S-2P W and S-2P Omicron BA.4/BA.5 recombinant proteins (group E). The antisera were harvested at 2 weeks after the third injection and subjected to neutralization assays with pseudoviruses of SARS-CoV-2 Wuhan strain (WT) and Omicron BA.4/BA.5 subvariant. Each dot represents neutralizing antibody titer of an individual serum sample. Bars indicate geometric mean titers (GMT) and error bars indicate 95% confidence intervals. The lower dotted line represents the lower limit of detection (100 in ID₉₀), and the upper dotted line represents the upper limit of detection (12800 in ID₉₀). *p<0.05.

### DETAILED DESCRIPTION OF THE PREFERRED EMBODIMENT

The present invention relates to an immunogenic composition against SARS-CoV-2. The immunogenic composition comprises an antigenic recombinant protein and an adjuvant containing an aluminum-containing adjuvant and/or an unmethylated cytosine-phosphate-guanosine (CpG) motif. The antigenic recombinant protein comprises a recombinant protein derived from the spike protein of SARS-CoV-2 Omicron subvariants, such as, but is not limited to, BA.1, BA.4/BA.5, BA.2.75, BA.2, BA.4.6. BF.7, BQ.1.1, BQ.1. CH.1.1, XBB, XBB.1, XBB.1.5. XBB.1.16, BA.2.86, EG.5, and EG.5.1.

The present invention further relates to a method for eliciting an immune response against severe acute respiratory syndrome coronavirus 2 (SARS-CoV-2) in a subject in need thereof, a method for protecting a subject in need thereof from infection with severe acute respiratory syndrome coronavirus 2 (SARS-CoV-2), and a method for preventing a subject in need thereof from contracting COVID-19 disease. The methods comprise administering to the subject at least one dose of the immunogenic composition of the present invention.

The present invention also relates to the immunogenic composition of the present invention for use in eliciting an immune response against a severe acute respiratory syndrome coronavirus 2 (SARS-CoV-2) in a subject in need thereof, for use in protecting a subject in need thereof from infection with severe acute respiratory syndrome coronavirus 2 (SARS-CoV-2), and for use in preventing a subject in need thereof from contracting COVID-19 disease caused by SARS-CoV-2.

The present invention also relates to use of the immunogenic composition of the present invention for the manufacture of a medicament for eliciting an immune response against a severe acute respiratory syndrome coronavirus 2 (SARS-CoV-2) in a subject in need thereof, for protecting a subject in need thereof from infection with severe acute respiratory syndrome coronavirus 2 (SARS-CoV-2), and for preventing a subject in need thereof from contracting COVID-19 disease.

### Definitions

All scientific and technical terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent or later-developed techniques which would be apparent to one of skill in the art. In addition, in order to more clearly and concisely describe the subject matter which is the invention, the following definitions are provided for certain terms which are used in the specification and appended claims.

As used herein, the singular form “a”, “an”, and “the” includes plural references unless indicated otherwise. For example, “an” excipient includes one or more excipients.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

The phrase “and/or,” as used herein in the specification and in the claims, should be understood to mean “either or both” of the elements so conjoined. i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims. “consisting of.” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein in the specification and in the claims, the phrase “at least one,” in reference to a unit of an element, should be understood to mean one or more than one unit of the element. Thus, as a non-limiting example, “at least one dose of an immunogenic composition” can refer, in one embodiment, to one or more than one (such as, two, three, or more) dose of the immunogenic composition.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

It should also be understood that, unless clearly indicated to the contrary, in any methods claimed herein that include more than one step or act, the order of the steps or acts of the method is not necessarily limited to the order in which the steps or acts of the method are recited.

As used interchangeably herein, the terms “polynucleotide” and “oligonucleotide” include single-stranded DNA (ssDNA), double-stranded DNA (dsDNA), single-stranded RNA (ssRNA) and double-stranded RNA (dsRNA), modified oligonucleotides and oligonucleosides or combinations thereof. The oligonucleotide can be linearly or circularly configured, or the oligonucleotide can contain both linear and circular segments. Oligonucleotides are polymers of nucleosides joined, generally, through phosphodiester linkages, although alternate linkages, such as phosphorothioate esters may also be used in oligonucleotides. A nucleoside consists of a purine (adenine (A) or guanine (G) or derivative thereof) or pyrimidine (thymine (T), cytosine (C) or uracil (U), or derivative thereof) base bonded to a sugar. The four nucleoside units (or bases) in DNA are called deoxyadenosine, deoxyguanosine, thymidine, and deoxycytidine. A nucleotide is a phosphate ester of a nucleoside.

As used herein, the term “severe acute respiratory syndrome coronavirus 2 (SARS-CoV-2)” refers to the strains and variants of coronavirus that cause coronavirus disease 2019 (COVID-19). SARS-CoV-2 is a positive-sense single-stranded RNA virus that is a member of the genus Betacoronavirus of the family Coronavirinae. The RNA sequence of SARS-CoV-2 is approximately 30,000 bases in length. Each SARS-CoV-2 virion is 50-200 nanometres in diameter. Like other coronaviruses, SARS-CoV-2 has four structural proteins, known as the S (spike), E (envelope), M (membrane), and N (nucleocapsid) proteins. The N protein holds the RNA genome, and the S. E, and M proteins together create the viral envelope. The S protein is the protein responsible for allowing the virus to attach to and fuse with the membrane of a host cell.

As used herein, the term “spike protein,” “S polypeptide,” “S protein,” “SARS-CoV-2 spike,” or “SARS-CoV-2 S protein.” which can be used interchangeably, refers to a surface structure glycoprotein on SARS CoV-2 and is responsible for allowing the virus to attach to and fuse with the membrane of a host cell. Each monomer of trimeric S protein is about 180-200 kDa, and contains a signal peptide at the N-terminus and two subunits, S1 and S2, mediating attachment and membrane fusion, respectively. The S1 subunit can be further divided into N-terminal domain (NTD), receptor-binding domain (RBD), and C-terminal domains (CTD1 and CTD2), whereas the S2 subunit contains fusion peptide (FP), fusion-peptide proximal region (FPPR), heptad repeat 1 (HR1), central helix (CH), connector domain (CD), heptad repeat 2 (HR2), transmembrane domain (TM), and cytoplasmic tail (CT). The junction between the S1 subunit and the S2 subunit is an S1/S2 cleavage site (S1/S2). Spike protein mainly enters human cells by binding to the receptor angiotensin converting enzyme 2 (ACE2).

As used interchangeably herein, the terms “S-2P” and “S-2P recombinant protein” refer to a recombinant protein of spike protein of a SARS-CoV-2 strain, in which the signal peptide (SP), the transmembrane domain (TM), and the cytoplasmic tail (CT) are removed, four residues at the S1/S2 cleavage site (S1/S2) are substituted with “GSAS” residues, the junction between the heptad repeat 1 (HR1) and the central helix (CH) is substituted with two proline residues, and a T4 fibritin trimerization motif (SEQ ID NO: 2) is added at the C-terminus. In some embodiments, an HRV3C protease cleavage sequence (SEQ ID NO: 3) is added to the C-terminus of the T4 fibritin trimerization motif for removing an 8×His tag, which is used for protein purification. Therefore, an “S-2P” or “S-2P recombinant protein” substantially consists of the S1 subunit and the S2 subunit without the transmembrane domain (TM) and the cytoplasmic tail (CT) of spike protein of a SARS-CoV-2 strain at the N-terminus, with a “GSAS” substitution at the S1/S2 cleavage site (S1/S2) and two proline substitutions at the junction between the heptad repeat 1 (HR1) and the central helix (CH), and a T4 fibritin trimerization motif (SEQ ID NO: 2) at the C-terminus. In some embodiments, the “S-2P” or “S-2P recombinant protein” has a cleaved HRV3C protease cleavage sequence (SEQ ID NO: 19) at the C-terminus of the T4 fibritin trimerization motif.

As used interchangeably herein, the terms “S-2P W,” “S-2P W recombinant protein.” and “S-2P recombinant protein derived from SARS-CoV-2 Wuhan strain” refer to an S-2P recombinant protein of SARS-CoV-2 Wuhan strain. Therefore, S-2P W recombinant protein substantially consists of the S1 subunit and the S2 subunit without the TM and the CT of spike protein of SARS-CoV-2 Wuhan strain at the N-terminus (i.e., residues 14-1208 of spike protein of SARS-CoV-2 Wuhan strain), with a “GSAS” substitution at the S1/S2 cleavage site (residues 682-685) and two proline substitutions at the junction (residues 986-987) between the HR1 and the CH, and a T4 fibritin trimerization motif (SEQ ID NO: 2) at the C-terminus. In some embodiments, the S-2P W recombinant protein has a cleaved HRV3C protease cleavage sequence (SEQ ID NO: 19) at the C-terminus of the T4 fibritin trimerization motif. In some embodiments, the S-2P W recombinant protein comprises an amino acid sequence at least 98%, 99%, or 100% identical to SEQ ID NO: 5 or 6.

As used herein, the term “Omicron variant,” also known as the lineage B.1.1.529, including all its sublineages (or subvariants), such as, but is not limited to, BA. 1, BA.4/BA.5, BA.2.75, BA.2, BA.4.6, BF.7, BQ.1.1, BQ.1, CH.1.1, XBB. XBB.1, XBB.1.5, XBB.1.16, BA.2.86, EG.5, and EG.5.1, refers to a variant of SARS-CoV-2 having a spike protein comprising at least the following common mutations compared to the spike protein of SARS-CoV-2 Wuhan strain (wild type; SEQ ID NO: 42): G142D, S373P, S375F, K417N, N440K, S477N, T478K, E484A, Q498R, N501Y, Y505H, D614G, H655Y, N679K, P681H, N764K, D796Y, Q954H, and N969K. The spike protein comprising the common mutations (G142D, S373P, S375F, K417N, N440K, S477N, T478K, E484A, Q498R, N501Y, Y505H, D614G, H655Y, N679K, P681H, N764K, D796Y, Q954H, and N969K) compared to the spike protein of SARS-CoV-2 Wuhan strain (SEQ ID NO: 42) has an amino acid sequence of SEQ ID NO: 43. As an example, the amino acid sequence of spike protein of SARS-CoV-2 Omicron BA.1 subvariant has the following substitutions compared to the spike protein of SARS-CoV-2 Wuhan strain (SEQ ID NO: 42): A67V, del69-70, T95I, G142D, del143-145, del211, L212I, ins214EPE, G339D, S371L, S373P, S375F, K417N, N440K, G446S, S477N. T478K, E484A, Q493R, G496S, Q498R, N501Y, Y505H, T547K, D614G, H655Y, N679K, P681H, N764K, D796Y, N856K, Q954H, N969K, and L981F, and the spike protein of SARS-CoV-2 Omicron BA.1 subvariant is 98.43% identical to SEQ ID NO: 43. As another example, the amino acid sequence of spike protein of SARS-CoV-2 Omicron BA.4/BA.5 subvariant (BA.4/BA.5, both possess identical spike protein sequences) has the following substitutions compared to the spike protein of SARS-CoV-2 Wuhan strain (SEQ ID NO: 42): T191, s24-26, A27S, del69-70, G142D, V213G, G339D, S371F, S373P, S375F, T376A, D405N, R408S, K417N, N440K, L452R, S477N, T478K, E484A, F486V, Q498R, N501Y, Y505H, D614G, H655Y, N679K, P681H, N764K, D796Y, Q954H, and N969K, and the spike protein of SARS-CoV-2 Omicron BA.4/BA.5 subvariant is 98.82% identical to SEQ ID NO: 43. Therefore, as used herein, an SARS-CoV-2 Omicron subvariant has a spike protein comprising at least the following common mutations compared to the spike protein of SARS-CoV-2 Wuhan strain (wild type; SEQ ID NO: 42): G142D, S373P, S375F, K417N, N440K, S477N, T478K, E484A, Q498R, N501Y Y505H, D614G, H655Y, N679K, P681H, N764K, D796Y, Q954H, and N969K, and preferably is at least 95%, 96% 97%, 98%, 99%, or 100% identical to SEQ ID NO: 43. For more mutations/substitutions of the amino acid sequence of S protein of different SARS-CoV-2 Omicron subvariants, please refer to https://viralzone.expasy.org/ or https://www.who.int/en/activities/tracking-SARS-CoV-2-variants/.

As used interchangeably herein, the terms “S-2P Omicron,” “S-2P Omicron recombinant protein,” and “S-2P recombinant protein derived from a SARS-CoV-2 Omicron subvariant” refer to an S-2P recombinant protein of a SARS-CoV-2 Omicron subvariant. Therefore, “S-2P Omicron” or “S-2P Omicron recombinant protein” substantially consists of the S1 subunit and the S2 subunit without the transmembrane domain (TM) and the cytoplasmic tail (CT) of spike protein of a SARS-CoV-2 Omicron subvariant at the N-terminus, with a “GSAS” substitution at the S1/S2 cleavage site (S1/S2) and two proline substitutions at the junction between the heptad repeat 1 (HR1) and the central helix (CH), and a T4 fibritin trimerization motif (SEQ ID NO: 2) at the C-terminus, and the spike protein of a SARS-CoV-2 Omicron subvariant comprises at least the following common mutations compared to the spike protein of SARS-CoV-2 Wuhan strain (SEQ ID NO: 42): G142D, S373P, S375F, K417N, N440K, S477N, T478K, E484A, Q498R, N501Y, Y505H, D614G, H655Y, N679K, P681H, N764K, D796Y, Q954H, and N969K. The “S-2P Omicron” or “S-2P Omicron recombinant protein” comprising the common mutations (G142D, S373P, S375F, K417N, N440K, S477N, T478K, E484A, Q498R, N501Y, Y505H, D614G, H655Y, N679K, P681H, N764K, D796Y, Q954H, and N969K) compared to the spike protein of SARS-CoV-2 Wuhan strain (SEQ ID NO: 42) has an amino acid sequence of SEQ ID NO: 44. In some embodiments, the “S-2P Omicron” or “S-2P Omicron recombinant protein” has a cleaved HRV3C protease cleavage sequence (SEQ ID NO: 19) at the C-terminus of the T4 fibritin trimerization motif, and therefore, the “S-2P Omicron” or “S-2P Omicron recombinant protein” comprising the common mutations (G142D, S373P, S375F, K417N, N440K. S477N, T478K, E484A, Q498R, N501Y, Y505H, D614G, H655Y, N679K, P681H, N764K, D796Y, Q954H, and N969K) compared to the spike protein of SARS-CoV-2 Wuhan strain (SEQ ID NO: 42) with the cleaved HRV3C protease cleavage sequence (SEQ ID NO: 19) at the C-terminus has an amino acid sequence of SEQ ID NO: 45. The Omicron subvariant may be any subvariant designated under Omicron variant, including but not limited to BA.1, BA.4/BA.5, BA.2.75, BA.2, BA.4.6, BF.7, BQ.1.1, BQ.1, CH.1.1, XBB, XBB.1, XBB.1.5, XBB.1.16, BA.2.86, EG.5, and EG.5.1.

In some embodiments, the S-2P Omicron recombinant protein is an S-2P recombinant protein of SARS-CoV-2 Omicron BA.1 subvariant, i.e., S-2P Omicron BA.1 or S-2P Omicron BA.1 recombinant protein. Therefore, the S-2P Omicron BA.1 recombinant protein substantially consists of the S1 subunit and the S2 subunit without the TM and the CT of spike protein of SARS-CoV-2 Omicron BA.1 subvariant at the N-terminus (i.e., residues 14-1205 of spike protein of SARS-CoV-2 Omicron BA.1 subvariant), with a “GSAS” substitution at the S1/S2 cleavage site (residues 679-682) and two proline substitutions at the junction (residues 983-984) between the HR1 and the CH, and a T4 fibritin trimerization motif (SEQ ID NO: 2) at the C-terminus. In some embodiments, the S-2P Omicron BA.1 recombinant protein comprises an amino acid sequence of SEQ ID NO: 14, which is 98.37% identical to SEQ ID NO: 44. In some embodiments, the S-2P Omicron BA.1 recombinant protein has a cleaved HRV3C protease cleavage sequence (SEQ ID NO: 19) at the C-terminus of the T4 fibritin trimerization motif, and comprises an amino acid sequence of SEQ ID NO: 15, which is 98.38% identical to SEQ ID NO: 45. In some embodiments, the S-2P Omicron BA.1 recombinant protein comprises an amino acid sequence at least 98%, 99%, or 100% identical to SEQ ID NO: 14 or 15.

In some embodiments, the S-2P Omicron recombinant protein is an S-2P recombinant protein of SARS-CoV-2 Omicron BA.4/BA.5 subvariant, i.e., S-2P Omicron BA.4/BA.5 or S-2P Omicron BA.4/BA.5 recombinant protein. Therefore, the S-2P Omicron BA.4/BA.5 recombinant protein substantially consists of the S1 subunit and the S2 subunit without the TM and the CT of spike protein of SARS-CoV-2 Omicron BA.4/BA.5 subvariant at the N-terminus (i.e., residues 14-1203 of spike protein of SARS-CoV-2 Omicron BA.4/BA.5 subvariant), with a “GSAS” substitution at the S1/S2 cleavage site (residues 677-680) and two proline substitutions at the junction (residues 981-982) between the HR1 and the CH, and a T4 fibritin trimerization motif (SEQ ID NO: 2) at the C-terminus. In some embodiments, the S-2P Omicron BA.4BA.5 recombinant protein comprises an amino acid sequence of SEQ ID NO: 17, which is 98.78% identical to SEQ ID NO: 44. In some embodiments, the S-2P Omicron BA.4/BA.5 recombinant protein has a cleaved HRV3C protease cleavage sequence (SEQ ID NO: 19) at the C-terminus of the T4 fibritin trimerization motif, and comprises an amino acid sequence of SEQ ID NO: 18, which is 98.78% identical to SEQ ID NO: 45. In some embodiments, the S-2P Omicron BA.4/BA.5 recombinant protein comprises an amino acid sequence at least 98%, 99%, or 100% identical to SEQ ID NO: 17 or 18.

In some embodiments, the S-2P Omicron recombinant protein is an S-2P recombinant protein of SARS-CoV-2 Omicron BA.2.75 subvariant, i.e., S-2P Omicron BA.2.75 or S-2P Omicron BA.2.75 recombinant protein. Therefore, the S-2P Omicron BA.2.75 recombinant protein substantially consists of the S1 subunit and the S2 subunit without the TM and the CT of spike protein of SARS-CoV-2 Omicron BA.2.75 subvariant at the N-terminus (i.e., residues 14-1205 of spike protein of SARS-CoV-2 Omicron BA.2.75 subvariant), with a “GSAS” substitution at the S1/S2 cleavage site (residues 679-682) and two proline substitutions at the junction (residues 983-984) between the HR1 and the CH, and a T4 fibritin trimerization motif (SEQ ID NO: 2) at the C-terminus. In some embodiments, the S-2P Omicron BA.2.75 recombinant protein comprises an amino acid sequence of SEQ ID NO: 20, which is 98.53% identical to SEQ ID NO: 44. In some embodiments, the S-2P Omicron BA.2.75 recombinant protein has a cleaved HRV3C protease cleavage sequence (SEQ ID NO: 19) at the C-terminus of the T4 fibritin trimerization motif, and comprises an amino acid sequence of SEQ ID NO: 21, which is 98.54% identical to SEQ ID NO: 45. In some embodiments, the S-2P Omicron BA. 2.75 recombinant protein comprises an amino acid sequence at least 98%, 99%, or 100% identical to SEQ ID NO: 20 or 21.

In some embodiments, the S-2P Omicron recombinant protein is an S-2P recombinant protein of SARS-CoV-2 Omicron BA.2 subvariant, i.e., S-2P Omicron BA.2 or S-2P Omicron BA.2 recombinant protein. Therefore, the S-2P Omicron BA.2 recombinant protein substantially consists of the S1 subunit and the S2 subunit without the TM and the CT of spike protein of SARS-CoV-2 Omicron BA.2 subvariant at the N-terminus (i.e., residues 14-1205 of spike protein of SARS-CoV-2 Omicron BA.2 subvariant), with a “GSAS” substitution at the S1/S2 cleavage site (residues 679-682) and two proline substitutions at the junction (residues 983-984) between the HR1 and the CH, and a T4 fibritin trimerization motif (SEQ ID NO: 2) at the C-terminus. In some embodiments, the S-2P Omicron BA.2 recombinant protein comprises an amino acid sequence of SEQ ID NO: 22, which is 99.02% identical to SEQ ID NO: 44. In some embodiments, the S-2P Omicron BA.2 recombinant protein has a cleaved HRV3C protease cleavage sequence (SEQ ID NO: 19) at the C-terminus of the T4 fibritin trimerization motif, and comprises an amino acid sequence of SEQ ID NO: 23, which is 99.03% identical to SEQ ID NO: 45. In some embodiments, the S-2P Omicron BA.2 recombinant protein comprises an amino acid sequence at least 98%, 99%, or 100% identical to SEQ ID NO: 22 or 23.

In some embodiments, the S-2P Omicron recombinant protein is an S-2P recombinant protein of SARS-CoV-2 Omicron BA.4.6 subvariant, i.e., S-2P Omicron BA.4.6 or S-2P Omicron BA.4.6 recombinant protein. Therefore, the S-2P Omicron BA.4.6 recombinant protein substantially consists of the S1 subunit and the S2 subunit without the TM and the CT of spike protein of SARS-CoV-2 Omicron BA.4.6 subvariant at the N-terminus (i.e., residues 14-1203 of spike protein of SARS-CoV-2 Omicron BA.4.6 subvariant), with a “GSAS” substitution at the S1/S2 cleavage site (residues 677-680) and two proline substitutions at the junction (residues 981-982) between the HR1 and the CH, and a T4 fibritin trimerization motif (SEQ ID NO: 2) at the C-terminus. In some embodiments, the S-2P Omicron BA.4.6 recombinant protein comprises an amino acid sequence of SEQ ID NO: 24, which is 98.61% identical to SEQ ID NO: 44. In some embodiments, the S-2P Omicron BA.4.6 recombinant protein has a cleaved HRV3C protease cleavage sequence (SEQ ID NO: 19) at the C-terminus of the T4 fibritin trimerization motif, and comprises an amino acid sequence of SEQ ID NO: 25, which is 98.62% identical to SEQ ID NO: 45. In some embodiments, the S-2P Omicron BA.4.6 recombinant protein comprises an amino acid sequence at least 98%, 99%, 100% identical to SEQ ID NO: 24 or 25.

In some embodiments, the S-2P Omicron recombinant protein is an S-2P recombinant protein of SARS-CoV-2 Omicron BF.7 subvariant. i.e., S-2P Omicron BF.7 or S-2P Omicron BF.7 recombinant protein. Therefore, the S-2P Omicron BF.7 recombinant protein substantially consists of the S1 subunit and the S2 subunit without the TM and the CT of spike protein of SARS-CoV-2 Omicron BF.7 subvariant at the N-terminus (i.e., residues 14-1203 of spike protein of SARS-CoV-2 Omicron BF.7 subvariant), with a “GSAS” substitution at the S1/S2 cleavage site (residues 677-680) and two proline substitutions at the junction (residues 981-982) between the HR1 and the CH, and a T4 fibritin trimerization motif (SEQ ID NO: 2) at the C-terminus. In some embodiments, the S-2P Omicron BF.7 recombinant protein comprises an amino acid sequence of SEQ ID NO: 26, which is 98.70% identical to SEQ ID NO: 44. In some embodiments, the S-2P Omicron BF.7 recombinant protein has a cleaved HRV3C protease cleavage sequence (SEQ ID NO: 19) at the C-terminus of the T4 fibritin trimerization motif, and comprises an amino acid sequence of SEQ ID NO: 27, which is 98.70% identical to SEQ ID NO: 45. In some embodiments, the S-2P Omicron BF.7 recombinant protein comprises an amino acid sequence at least 98%, 99%, or 100% identical to SEQ ID NO: 26 or 27.

In some embodiments, the S-2P Omicron recombinant protein is an S-2P recombinant protein of SARS-CoV-2 Omicron BQ.1 subvariant, i.e., S-2P Omicron BQ.1 or S-2P Omicron BQ.1 recombinant protein. Therefore, the S-2P Omicron BQ.1 recombinant protein substantially consists of the S1 subunit and the S2 subunit without the TM and the CT of spike protein of SARS-CoV-2 Omicron BQ.1 subvariant at the N-terminus (i.e., residues 14-1203 of spike protein of SARS-CoV-2 Omicron BQ.1 subvariant), with a “GSAS” substitution at the S1/S2 cleavage site (residues 677-680) and two proline substitutions at the junction (residues 981-982) between the HR1 and the CH, and a T4 fibritin trimerization motif (SEQ ID NO: 2) at the C-terminus. In some embodiments, the S-2P Omicron BQ.1 recombinant protein comprises an amino acid sequence of SEQ ID NO: 28, which is 98.61% identical to SEQ ID NO: 44. In some embodiments, the S-2P Omicron BQ.1 recombinant protein has a cleaved HRV3C protease cleavage sequence (SEQ ID NO: 19) at the C-terminus of the T4 fibritin trimerization motif, and comprises an amino acid sequence of SEQ ID NO: 29, which is 98.62% identical to SEQ ID NO: 45. In some embodiments, the S-2P Omicron BQ.1 recombinant protein comprises an amino acid sequence at least-98%, 99%, or 100% identical to SEQ ID NO: 28 or 29.

In some embodiments, the S-2P Omicron recombinant protein is an S-2P recombinant protein of SARS-CoV-2 Omicron BQ.1.1 subvariant, i.e., S-2P Omicron BQ.1.1 or S-2P Omicron BQ.1.1 recombinant protein. Therefore, the S-2P Omicron BQ.1.1 recombinant protein substantially consists of the S1 subunit and the S2 subunit without the TM and the CT of spike protein of SARS-CoV-2 Omicron BQ.1.1 subvariant at the N-terminus (i.e., residues 14-1203 of spike protein of SARS-CoV-2 Omicron BQ.1.1 subvariant), with a “GSAS” substitution at the S1/S2 cleavage site (residues 677-680) and two proline substitutions at the junction (residues 981-982) between the HR1 and the CH, and a T4 fibritin trimerization motif (SEQ ID NO: 2) at the C-terminus. In some embodiments, the S-2P Omicron BQ.1.1 recombinant protein comprises an amino acid sequence of SEQ ID NO: 30, which is 98.53% identical to SEQ ID NO: 44. In some embodiments, the S-2P Omicron BQ.1.1 recombinant protein has a cleaved HRV3C protease cleavage sequence (SEQ ID NO: 19) at the C-terminus of the T4 fibritin trimerization motif, and comprises an amino acid sequence of SEQ ID NO: 31, which is 98.54% identical to SEQ ID NO: 45. In some embodiments, the S-2P Omicron BQ.1.1 recombinant protein comprises an amino acid sequence at least 98%, 99%, or 100% identical to SEQ ID NO: 30 or 31.

In some embodiments, the S-2P Omicron recombinant protein is an S-2P recombinant protein of SARS-CoV-2 Omicron XBB.1.5 subvariant, i.e., S-2P Omicron XBB.1.5 or S-2P Omicron XBB.1.5 recombinant protein. Therefore, the S-2P Omicron XBB.1.5 recombinant protein substantially consists of the S1 subunit and the S2 subunit without the TM and the CT of spike protein of SARS-CoV-2 Omicron XBB.1.5 subvariant at the N-terminus (i.e., residues 14-1204 of spike protein of SARS-CoV-2 Omicron XBB.1.5 subvariant), with a “GSAS” substitution at the S1/S2 cleavage site (residues 678-681) and two proline substitutions at the junction (residues 982-983) between the HR1 and the CH, and a T4 fibritin trimerization motif (SEQ ID NO: 2) at the C-terminus. In some embodiments, the S-2P Omicron XBB.1.5 recombinant protein comprises an amino acid sequence of SEQ ID NO: 32, which is 98.13% identical to SEQ ID NO: 44. In some embodiments, the S-2P Omicron XBB.1.5 recombinant protein has a cleaved HRV3C protease cleavage sequence (SEQ ID NO: 19) at the C-terminus of the T4 fibritin trimerization motif, and comprises an amino acid sequence of SEQ ID NO: 33, which is 98.13% identical to SEQ ID NO: 45. In some embodiments, the S-2P Omicron XBB.1.5 recombinant protein comprises an amino acid sequence at least 98%, 99%, or 100% identical to SEQ ID NO: 32 or 33.

In some embodiments, the S-2P Omicron recombinant protein is an S-2P recombinant protein of SARS-CoV-2 Omicron XBB.1 subvariant, i.e., S-2P Omicron XBB.1 or S-2P Omicron XBB.1 recombinant protein. Therefore, the S-2P Omicron XBB.1 recombinant protein substantially consists of the S1 subunit and the S2 subunit without the TM and the CT of spike protein of SARS-CoV-2 Omicron XBB.1 subvariant at the N-terminus (i.e., residues 14-1204 of spike protein of SARS-CoV-2 Omicron XBB.1 subvariant), with a “GSAS” substitution at the S1/S2 cleavage site (residues 678-681) and two proline substitutions at the junction (residues 982-983) between the HR1 and the CH, and a T4 fibritin trimerization motif (SEQ ID NO: 2) at the C-terminus. In some embodiments, the S-2P Omicron XBB.1 recombinant protein comprises an amino acid sequence of SEQ ID NO: 34, which is 98.13% identical to SEQ ID NO: 44. In some embodiments, the S-2P Omicron XBB.1 recombinant protein has a cleaved HRV3C protease cleavage sequence (SEQ ID NO: 19) at the C-terminus of the T4 fibritin trimerization motif, and comprises an amino acid sequence of SEQ ID NO: 35, which is 98.13% identical to SEQ ID NO: 45. In some embodiments, the S-2P Omicron XBB.1 recombinant protein comprises an amino acid sequence at least 98%, 99%, or 100% identical to SEQ ID NO: 34 or 35.

In some embodiments, the S-2P Omicron recombinant protein is an S-2P recombinant protein of SARS-CoV-2 Omicron XBB.1.16 subvariant, i.e., S-2P Omicron XBB.1.16 or S-2P Omicron XBB.1.16 recombinant protein. Therefore, the S-2P Omicron XBB.1.16 recombinant protein substantially consists of the S1 subunit and the S2 subunit without the TM and the CT of spike protein of SARS-CoV-2 Omicron XBB.1.16 subvariant at the N-terminus (i.e., residues 14-1204 of spike protein of SARS-CoV-2 Omicron XBB.1.16 subvariant), with a “GSAS” substitution at the S1/S2 cleavage site (residues 678-681) and two proline substitutions at the junction (residues 982-983) between the HR1 and the CH, and a T4 fibritin trimerization motif (SEQ ID NO: 2) at the C-terminus. In some embodiments, the S-2P Omicron XBB.1.16 recombinant protein comprises an amino acid sequence of SEQ ID NO: 36, which is 98.04% identical to SEQ ID NO: 44. In some embodiments, the S-2P Omicron XBB.1.16 recombinant protein has a cleaved HRV3C protease cleavage sequence (SEQ ID NO: 19) at the C-terminus of the T4 fibritin trimerization motif, and comprises an amino acid sequence of SEQ ID NO: 37, which is 98.05% identical to SEQ ID NO: 45. In some embodiments, the S-2P Omicron XBB.1.16 recombinant protein comprises an amino acid sequence at least 98%, 99%, or 100% identical to SEQ ID NO: 36 or 37.

In some embodiments, the S-2P Omicron recombinant protein is an S-2P recombinant protein of SARS-CoV-2 Omicron BA.2.86 subvariant, i.e., S-2P Omicron BA.2.86 or S-2P Omicron BA.2.86 recombinant protein. Therefore, the S-2P Omicron BA.2.86 recombinant protein substantially consists of the S1 subunit and the S2 subunit without the TM and the CT of spike protein of SARS-CoV-2 Omicron BA.2.86 subvariant at the N-terminus (i.e., residues 14-1200 of spike protein of SARS-CoV-2 Omicron BA.2.86 subvariant), with a “GSAS” substitution at the S1/S2 cleavage site (residues 674-677) and two proline substitutions at the junction (residues 978-979) between the HR1 and the CH, and a T4 fibritin trimerization motif (SEQ ID NO: 2) at the C-terminus. In some embodiments, the S-2P Omicron BA.2.86 recombinant protein comprises an amino acid sequence of SEQ ID NO: 38, which is 96.50% identical to SEQ ID NO: 44. In some embodiments, the S-2P Omicron BA.2.86 recombinant protein has a cleaved HRV3C protease cleavage sequence (SEQ ID NO: 19) at the C-terminus of the T4 fibritin trimerization motif, and comprises an amino acid sequence of SEQ ID NO: 39, which is 96.51% identical to SEQ ID NO: 45. In some embodiments, the S-2P Omicron BA.2.86 recombinant protein comprises an amino acid sequence at least 98%, 99%, or 100% identical to SEQ ID NO: 38 or 39.

In some embodiments, the S-2P Omicron recombinant protein is an S-2P recombinant protein of SARS-CoV-2 Omicron EG.5.1 subvariant, i.e., S-2P Omicron EG.5.1 or S-2P Omicron EG.5.1 recombinant protein. Therefore, the S-2P Omicron EG.5.1 recombinant protein substantially consists of the S1 subunit and the S2 subunit without the TM and the CT of spike protein of SARS-CoV-2 Omicron EG.5.1 subvariant at the N-terminus (i.e., residues 14-1204 of spike protein of SARS-CoV-2 Omicron EG.5.1 subvariant), with a “GSAS” substitution at the S1/S2 cleavage site (residues 678-681) and two proline substitutions at the junction (residues 982-983) between the HR1 and the CH, and a T4 fibritin trimerization motif (SEQ ID NO: 2) at the C-terminus. In some embodiments, the S-2P Omicron EG.5.1 recombinant protein comprises an amino acid sequence of SEQ ID NO: 40, which is 97.96% identical to SEQ ID NO: 44. In some embodiments, the S-2P Omicron EG.5.1 recombinant protein has a cleaved HRV3C protease cleavage sequence (SEQ ID NO: 19) at the C-terminus of the T4 fibritin trimerization motif, and comprises an amino acid sequence of SEQ ID NO: 41, which is 97.97% identical to SEQ ID NO: 45. In some embodiments, the S-2P Omicron EG.5.1 recombinant protein comprises an amino acid sequence at least 98%, 99%, or 100% identical to SEQ ID NO: 40 or 41.

As used interchangeably herein, the terms “COVID-19 vaccine” and “immunogenic composition against severe acute respiratory syndrome coronavirus 2 (SARS-CoV-2)” refer to a composition for stimulating or eliciting an immune response against a SARS-CoV-2. The immune response includes, but not limited to, production of neutralizing antibodies against SARS-CoV-2 and Th1-skewed immune response. In some embodiments, the COVID-19 vaccine is a COVID-19 vaccine administrated to a subject via intramuscular injection, and one dose of the COVID-19 vaccine for human contains 5 μg, 15 μg, or 25 μg S-2P recombinant protein adjuvanted with 750 μg CpG 1018 adjuvant and 375 μg (Al³⁺ equivalent to weight) aluminum hydroxide. In some embodiments, one dose of the COVID-19 vaccine for rodents (e.g., mice, rats, and hamsters) contains ⅕ volume of one dose of the COVID-19 vaccine for human.

As used herein, the term “an immunogenic composition derived from SARS-CoV-2 Wuhan strain” refers to a composition for stimulating or eliciting an immune response against a SARS-CoV-2 and that the composition contains an antigen derived from at least a fragment of spike protein of SARS-CoV-2 Wuhan strain. In some embodiments, the at least a fragment of spike protein of SARS-CoV-2 Wuhan strain is the S-2P W recombinant protein comprising a polypeptide sequence at least 98%, 99%, or 100% identical to SEQ ID NO: 5 or 6. In some embodiments, the antigen derived from at least a fragment of spike protein of SARS-CoV-2 Wuhan strain is a polynucleotide encoding the S-2P W recombinant protein comprising a polypeptide sequence at least 98%, 99%, or 100% identical to SEQ ID NO: 5 or 6. In some embodiments, the antigen derived from at least a fragment of spike protein of SARS-CoV-2 Wuhan strain is a polypeptide of the S-2P W recombinant protein comprising a polypeptide sequence at least 98%, 99%, or 100% identical to SEQ ID NO: 5 or 6. In some embodiments, the immunogenic composition derived from SARS-CoV-2 Wuhan strain comprises a polypeptide sequence at least 98%, 99%, or 100% identical to SEQ ID NO: 5 or 6, and an adjuvant selected from the group consisting of an aluminum-containing adjuvant, an unmethylated cytosine-phosphate-guanosine (CpG) motif, and a combination thereof. In some embodiments, the immunogenic composition derived from SARS-CoV-2 Wuhan strain for human contains 5 μg, 15 μg, or 25 μg S-2P W recombinant protein adjuvanted with 750 μg CpG 1018 adjuvant and 375 μg (Al³⁺ equivalent to weight) aluminum hydroxide.

As used herein, the terms “adjuvant” refers to a substance which, when added to a composition comprising an antigen, nonspecifically enhances or potentiates an immune response to the antigen in the recipient upon exposure.

As used herein, the terms “aluminum-containing adjuvant” refers to an adjuvant including aluminum. In some embodiments, the aluminum-containing adjuvant includes, but not limited to, aluminum hydroxide, aluminum oxyhydroxide, aluminum hydroxide gel, aluminum phosphate, aluminum phosphate gel, aluminum hydroxyphosphate, aluminum hydroxyphosphate sulfate, amorphous aluminum hydroxyphosphate sulfate, potassium aluminum sulfate, aluminum monostearate or a combination thereof. In some embodiments, the aluminum-containing adjuvant is an aluminum-containing adjuvant approved for administration to humans by the FDA. In some embodiments, the aluminum-containing adjuvant is an aluminum hydroxide adjuvant approved for administration to humans by the FDA. In some embodiments, the aluminum-containing adjuvant is an aluminum phosphate adjuvant approved for administration to humans by the FDA.

As used herein, the terms “unmethylated cytosine-phosphate-guanosine (CpG) motif” refers to a CpG-containing oligonucleotide in which the C is unmethylated, and which contributes to a measurable immune response as measured in vitro, in vivo, and/or ex vivo. In some embodiments, the CpG-containing oligonucleotide contains palindromic hexamers following the general formula of: 5′-purine-purine-CG-pyrimidine-pyrimidine-3′. In some preferred embodiments, the unmethylated cytosine-phosphate-guanosine (CpG) motif has an oligonucleotide of SEQ ID NO: 8 (5′-TGACTGTGAACGTTCGAGATGA-3′) in which the Cs of the CGs are unmethylated. In some embodiments, the CpG-containing oligonucleotide contains TCG in which the C is unmethylated, and which is from 8 to 100 nucleotides, preferably 8 to 50 nucleotides, or preferably 8 to 25 nucleotides in length. In some preferred embodiments, the unmethylated cytosine-phosphate-guanosine (CpG) motif has an oligonucleotide of SEQ ID NO: 9 (5′-TCGTCGTTTTGTCGTTTTGTCGTT-3′) in which the Cs of the TCGs are unmethylated. Examples of the unmethylated cytosine-phosphate-guanosine (CpG) motif further includes, but not limited to, 5′-GGTGCATCGATGCAGGGG GG-3′ (SEQ ID NO: 10). 5′-TCCATGGACGTTCCTGAGCGTT-3′ (SEQ ID NO: 11), 5′-TCGTCGTTCGAACGACGTTGAT-3′ (SEQ ID NO: 12), and 5′-TCGTCGACGATCGGC GCGCGCCG-3′ (SEQ ID NO: 13). The CpG-containing oligonucleotide described herein are in their pharmaceutically acceptable salt form unless otherwise indicated. In one preferred embodiment, the CpG-containing oligonucleotides are in the sodium salt form.

An “effective amount” or a “sufficient amount” of a substance is that amount sufficient to effect beneficial or desired results, including clinical results, and, as such, an “effective amount” depends upon the context in which it is being applied. In the context of administering an immunogenic composition, an effective amount contains sufficient adjuvant and SARS-CoV-2 S-2P Omicron recombinant protein to elicit an immune response. An effective amount can be administered in one or more doses.

The terms “individual” and “subject” refer to mammals. “Mammals” include, but are not limited to, humans, non-human primates (e.g., monkeys), farm animals, sport animals, rodents (e.g., mice and rats) and pets (e.g., dogs and cats).

The term “dose” as used herein in reference to an immunogenic composition refers to a measured portion of the immunogenic composition taken by (administered to or received by) a subject at any one time.

The terms “isolated” and “purified” as used herein refers to a material that is removed from at least one component with which it is naturally associated (e.g., removed from its original environment). The term “isolated,” when used in reference to a recombinant protein, refers to a protein that has been removed from the culture medium of the host cell that produced the protein.

“Stimulation” of a response or parameter includes eliciting and/or enhancing that response or parameter when compared to otherwise same conditions except for a parameter of interest, or alternatively, as compared to another condition (e.g., increase in TLR-signaling in the presence of a TLR agonist as compared to the absence of the TLR agonist). For example, “stimulation” of an immune response means an increase in the response. Depending upon the parameter measured, the increase may be from 5-fold to 500-fold or over, or from 5, 10, 50, or 100-fold to 500, 1,000, 5,000, or 10,000-fold.

As used herein the term “immunization” refers to a process that increases a mammalian subject's reaction to antigen and therefore improves its ability to resist or overcome infection.

The term “vaccination” as used herein refers to the introduction of vaccine into a body of a mammalian subject.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

### Example 1 Preparation of Immunogenic Compositions Against SARS-CoV-2

Construct of S-2P recombinant protein derived from SARS-CoV-2 Wuhan strain (S-2P W). A plasmid having a polynucleotide encoding the residues 1-1208 of S protein of SARS-CoV-2 Wuhan-Hu-1 strain (wild type, GenBank accession number: MN908947) with proline substitutions at residues 986 and 987, a “GSAS” substitution at the furin cleavage site (residues 682-685) (SEQ ID NO: 1) and a C-terminal T4 fibritin trimerization motif (SEQ ID NO: 2), an HRV3C protease cleavage site (SEQ ID NO: 3), an 8×His Tag, and a Twin-Strep Tag (SEQ ID NO: 4) was transfected into ExpiCHO-S cells (Thermo Fisher Scientific, Waltham. MA, USA).

Construct of S-2P recombinant protein derived from SARS-CoV-2 Omicron BA.1 subvariant (S-2P Omicron BA.1). The amino acid sequence of S protein of SARS-CoV-2 Omicron BA.1 subvariant has the following substitutions compared to the S protein of SARS-CoV-2 Wuhan-Hu-1 strain (wild type): A67V, A69-70, T95I, G142D, A143-145, A211, L212I, ins214EPE, G339D, S371L, S373P, S375F, K417N, N440K, G446S, S477N, T478K, E484A. Q493R, G496S, Q498R. N501Y, Y505H, T547K, D614G, H655Y. N679K, P681H, N764K, D796Y, N856K. Q954H, N969K. and L981F (https://www.cdc.gov/coronavirus/2019-ncov/variants/variant-info.html). A plasmid having a polynucleotide encoding the residues 1-1205 of the S protein of SARS-CoV-2 Omicron BA.1 subvariant with proline substitutions at residues 983 and 984, a “GSAS” substitution at the furin cleavage site (residues 679-682) (SEQ ID NO: 13) and a C-terminal T4 fibritin trimerization motif (SEQ ID NO: 2), an HRV3C protease cleavage site (SEQ ID NO: 3), an 8×His Tag, and a Twin-Strep Tag (SEQ ID NO: 4) was transfected into ExpiCHO-S cells (Thermo Fisher Scientific, Waltham, MA, USA).

Construct of S-2P recombinant protein derived from SARS-CoV-2 Omicron BA.4/BA.5 subvariant (S-2P Omicron BA.4/BA.5). The amino acid sequence of S protein of SARS-CoV-2 Omicron BA.4/BA.5 subvariant has the following substitutions compared to the S protein of SARS-CoV-2 Wuhan-Hu-1 strain (wild type): T191, del24-26, A27S, del69-70, G142D, V213G, G339D, S371F, S373P, S375F, T376A, D405N, R408S, K417N, N440K, L452R, S477N, T478K, E484A, F486V, Q498R, N501Y, Y505H, D614G, H655Y, N679K. P681H. N764K, D796Y, Q954H, and N969K (https://www.cdc.gov/coronavirus/2019-ncov/variants/variant-info.html). A plasmid having a polynucleotide encoding the residues 1-1203 of the S protein of SARS-CoV-2 Omicron BA.4/BA.5 subvariant with proline substitutions at residues 981 and 982, a “GSAS” substitution at the furin cleavage site (residues 677-680) (SEQ ID NO: 16) and a C-terminal T4 fibritin trimerization motif (SEQ ID NO: 2), an HRV3C protease cleavage site (SEQ ID NO: 3), an 8×His Tag, and a Twin-Strep Tag (SEQ ID NO: 4) was transfected into ExpiCHO-S cells (Thermo Fisher Scientific, Waltham, MA, USA).

Cell culture was harvested after 6 days, and protein was purified from the supernatant using Strep-Tactin resin (IBA Lifesciences, Gottingen, Germany). HRV3C protease (1% wt/wt) was added to the protein and the reaction was incubated overnight at 4° C. The digested protein was further purified using a Superose 6 16/70 column (GE Healthcare Biosciences, Chicago, IL, USA). The purified S-2P W recombinant protein (SEQ ID NO: 5 or 6), the purified S-2P Omicron BA.1 recombinant protein (SEQ ID NO: 14 or 15), and/or the purified S-2P Omicron BA.4/BA.5 recombinant protein (SEQ ID NO: 17 or 18) was then formulated with an unmethylated CpG motif (CpG 1018, SEQ ID NO: 7) and/or aluminum-containing adjuvant, such as aluminum hydroxide (Al(OH)₃) as the immunogenic compositions against SARS-CoV-2.

### Example 2 Animal Trial

This example provides a description of preclinical studies to assess the immunogenicity of the immunogenic compositions obtained from Example 1 against SARS-CoV-2 Omicron variant in mice.

### Materials and Methods

Mouse immunizations. Female BALB/c mice aged 6-9 weeks were obtained from BioLASCO Taiwan Co., Ltd. (Yilan County, Taiwan). The mice were randomized from different litters into 6 groups (n=5 for each group) and immunized with the regimens shown in Table 1. All the mice were immunized three times (50 μL intramuscularly in each of the left and right quadriceps femoris muscles per mouse, for a total of 100 μL per dose) on days 1, 22, and 43. Serum samples were harvested on days 36 and 57 (i.e. 2 weeks after the second and third immunizations, respectively) and were subjected to pseudovirus neutralization assay.

TABLE 1
Vaccine regimens of the animal trial in Example 2

| | Vaccine used | | |
|---|---|---|---|
| | 1st | 2nd | 3rd |
| | immunization | immunization | immunization |
| Groups | (Day 1) | (Day 22) | (Day 43) |
| A: W + W + W | W | W | W |
| B: W + W + BA.1 | W | W | BA.1 |
| C: W + W + BA.4/BA.5 | W | W | BA.4/BA.5 |
| D: W + W + (W + BA.1) | W | W | (W + BA.1) |
| E: W + W + (W + BA.4/BA.5) | W | W | (W + BA.4/BA.5) |
| F: Adjuvant control | Adjuvant only | Adjuvant only | Adjuvant only |

W: immunogenic composition containing 3 μg S-2P W recombinant protein adjuvanted with 75 μg aluminum hydroxide and 150 μg CpG-1080 per 100 μL.
BA.1: immunogenic composition containing 3 μg S-2P Omicron BA.1 recombinant protein adjuvanted with 75 μg aluminum hydroxide and 150 μg CpG-1080 per 100 μL.
BA.4/BA.5: immunogenic composition containing 3 μg S-2P Omicron BA.4/BA.5 recombinant protein adjuvanted with 75 μg aluminum hydroxide and 150 μg CpG-1080 per 100 μL.
(W + BA.1): immunogenic composition containing a bivalent mixture of 1.5 μg S-2P W recombinant protein and 1.5 μg S-2P Omicron BA.1 recombinant protein adjuvanted with 75 μg aluminum hydroxide and 150 μg CpG-1080 per 100 μL.
(W + BA.4/BA.5): immunogenic composition containing a bivalent mixture of 1.5 μg S-2P W recombinant protein and 1.5 μg S-2P Omicron BA.4/BA.5 recombinant protein adjuvanted with 75 μg aluminum hydroxide and 150 μg CpG-1080 per 100 μL.
Adjuvant only: Solution containing 75 μg aluminum hydroxide and 150 μg CpG-1080 per 100 μL.

Pseudovirus production and titration. To produce SARS-CoV-2 pseudovirus, a plasmid expressing full-length SARS-CoV-2 spike protein was co-transfected into HEK293T cells with packaging and reporter plasmids pCMVΔ8.91 and pLAS2w.FLuc.Ppuro (RNAi Core, Academia Sinica), using TransIT-LT1 transfection reagent (Mirus Bio). The plasmid expresses the full-length spike protein of SARS-CoV-2 Omincron BA.4/BA.5 subvariant having the following substitutions compared to the S protein of Wuhan-Hu-1 strain (wild-type; GenBank Accession No. MN908947): T191, del24-26, A27S, del69-70, G142D, V213G, G339D, S371F, S373P, S375F, T376A, D405N, R408S, K417N, N440K, L452R, S477N, T478K, E484A, F486V, Q498R, N501Y, Y505H, D614G, H655Y, N679K, P681H, N764K, D796Y. Q954H, and N969K. Mock pseudoviruses were produced by omitting the p2019-nCoV spike (WT). Seventy-two (72) hours post-transfection, supernatants were collected, filtered, and frozen at −80° C. The transduction unit (TU) of SARS-CoV-2 pseudotyped lentivirus was estimated by using cell viability assay in response to the limited dilution of lentivirus. In brief, HEK-293 T cells stably expressing human ACE2 gene were plated on 96-well plate 1 day before lentivirus transduction. For the titration of pseudovirus, different amounts of pseudovirus were added into the culture medium containing polybrene. Spin infection was carried out at 1100×g in 96-well plate for 30 minutes at 37° C. After incubating cells at 37° C. for 16 hours, the culture media containing virus and polybrene were removed and replaced with fresh complete DMEM containing 2.5 μg/ml puromycin. After treating with puromycin for 48 hours, the culture media were removed and cell viability was detected by using 10% AlarmaBlue reagents according to manufacturer's instruction. The survival rate of uninfected cells (without puromycin treatment) was set as 100%. The virus titer (transduction units) was determined by plotting the survival cells versus diluted viral dose.

Pseudovirus-based neutralization assay. HEK293-hAce2 cells (2×10⁴ cells/well) were seeded in 96-well white isoplates and incubated for overnight. Sera were heated at 56° C. for 30 minutes to inactivate complement and diluted in MEM supplemented with 2% FBS at an initial dilution factor of 100, and then twofold serial dilutions were carried out (for a total of 8 dilution steps to a final dilution of 1:25,600). The diluted sera were mixed with an equal volume of pseudovirus (1000 TU) and incubated at 37° C. for 1 hour before adding to the plates with cells. After the 1-hour incubation, the culture medium was replaced with 50 μL of fresh medium. On the following day, the culture medium was replaced with 100 μL of fresh medium. Cells were lysed at 72 hours post infections and relative luciferase units (RLU) were measured. The luciferase activity was detected by Tecan i-control (Infinite 500). The 90% inhibition dilution titers (ID₉₀) were calculated considering uninfected cells as 100% neutralization and cells transduced with only virus as 0% neutralization. Reciprocal ID₉₀ geometric mean titers (GMT) were determined as ID₉₀ titer.

Statistical analysis. Prism 6.01 (GraphPad Software Inc., San Diego, CA, USA) was used for statistical analysis. Kruskal-Wallis with corrected Dunn's multiple comparisons test was used to calculate significance. * p<0.05, ** p<0.01, *** p<0.001, **** p<0.0001.

### Results

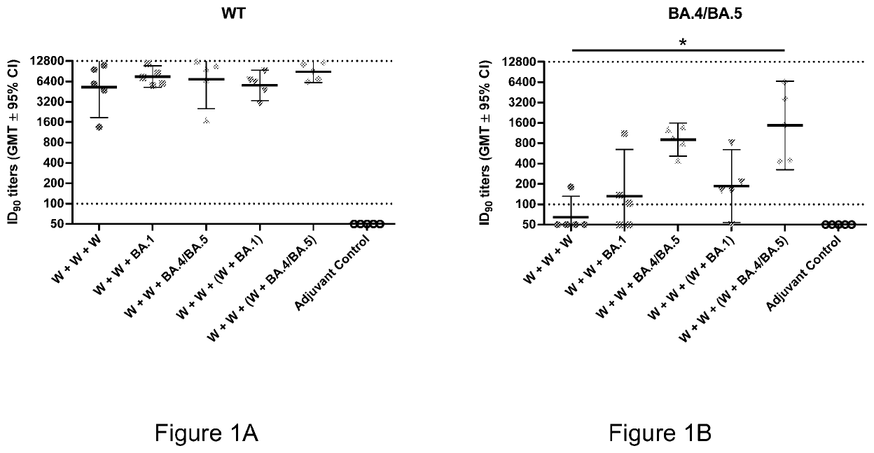

Administration of two doses of'S-2P W recombinant protein followed by one dose of immunogenic composition containing S-2P recombinant proteins derived from SARS-CoV-2 Omicron subvariants to mice induced higher level of neutralizing antibodies against SARS-CoV-2 Omicron variant than administration of three doses of S-2P W recombinant protein. Mice were divided into 6 groups receiving two doses of adjuvanted S-2P W recombinant protein followed by one dose of adjuvanted S-2P recombinant proteins (S-2P W, S-2P Omicron BA.1, S-2P Omicron BA.4/BA.5, a bivalent mixture of S-2P W and S-2P Omicron BA.1, or a bivalent mixture of S-2P w and S-2P Omicron BA.4/BA.5) at 21 days apart. No mortality, abnormality of clinical signs, differences in body weight changes, body temperature, nor food consumption were observed after vaccination among the 6 groups, indicating multiple doses of S-2P recombinant proteins (either S-2P W, S-2P Omicron BA.1, and/or S-2P Omicron BA.4/BA.5) do not result in systemic adverse effects in mice. Fourteen (14) days after the third immunization, neutralizing antibody titers were analyzed, and the results are shown in FIGS. 1A and 1B.

As shown in FIG. 1A, all vaccine combinations of the S-2P recombinant proteins (groups A to E) induce equally high neutralization antibody titers against SARS-CoV-2 Wuhan strain (wild type, WT). Administration of two doses of S-2P W recombination protein boosted with the bivalent mixture of S-2P W and S-2P Omicron BA.4/BA.5 recombinant proteins (group E) achieved the highest numerical GMT (8946 [95% CI: 6211-12886]), and administration of two doses of S-2P W recombination protein boosted with S-2P Omicron BA.1 and S-2P Omicron BA.4/BA.5 recombinant proteins (groups B and C) resulted in GMT of 7581 [95% CI: 5246-10955] and 6942 [95% CI: 2517-19145], respectively. Administration of three doses of S-2P W recombination protein (group A) resulted in the lowest GMT out of all treatment groups (5274 [95% CI: 1877-14824]), although the GMTs are not significantly different among all the treatment groups (groups A to E) (p>0.05).

As shown in FIG. 1B, administration of three doses of S-2P W recombination protein (group A) was ineffective against SARS-CoV-2 Omicron BA.4/BA.5 pseudovirus with the GMT below the lower limit of detection (64.6 [95% CI: 31.7-131.5]). Boosting with either S-2P Omicron BA.1 recombinant protein (group B) or the bivalent mixture of S-2P W and S-2P Omicron BA.1 recombinant proteins (group D) after two doses of S-2P W recombination protein slightly increased the GMTs (132.2 [95% CI: 27.1-645.8] and 186.3 [95% CI: 54-642.7], respectively). A booster dose of S-2P Omicron BA.4/BA.5 recombinant protein after two doses of S-2P W recombination protein (group C) had a higher increase in GMT of 907.9 [95% CI: 519.4-1587], and a booster dose of the bivalent mixture of S-2P W and S-2P Omicron BA.4/BA.5 recombinant proteins after two doses of S-2P W recombination protein (group E) significantly induced the highest neutralization titer (p<0.05) against SARS-CoV-2 Omicron BA.4/BA.5 pseudovirus with a GMT of 1473 [95% CI: 326.4-6645]. The results of the animal study indicate that the immunogenic compositions containing S-2P recombinant proteins derived from SARS-CoV-2 Omicron subvariants (either BA.1 or BA.4/BA.5) can be used to increase immunity against SARS-CoV-2 Omicron variant.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. An immunogenic composition against a severe acute respiratory syndrome coronavirus 2 (SARS-CoV-2), comprising an antigenic recombinant protein and an adjuvant selected from the group consisting of an aluminum-containing adjuvant, an unmethylated cytosine-phosphate-guanosine (CpG) motif, and a combination thereof, wherein the antigenic recombinant protein substantially consisting of an S1 subunit and an S2 subunit without a transmembrane domain (TM) and a cytoplasmic tail (CT) of spike protein of a SARS-CoV-2 Omicron subvariant at a N-terminus, with a “GSAS” substitution at an S1/S2 cleavage site (S1/S2) and two proline substitutions at a junction between a heptad repeat 1 (HR1) and a central helix (CH), and a C-terminal T4 fibritin trimerization motif of SEQ ID NO: 2, with or without a cleaved HRV3C protease cleavage sequence of SEQ ID NO: 19 at a C-terminus, and wherein the spike protein of a SARS-CoV-2 Omicron subvariant comprises at least the following common mutations compared to spike protein of SARS-CoV-2 Wuhan strain (SEQ ID NO: 42): G142D, S373P, S375F, K417N, N440K, S477N, T478K, E484A, Q498R, N501Y, Y505H, D614G, H655Y, N679K, P681H, N764K, D796Y, Q954H, and N969K.

2. The immunogenic composition of claim 1, wherein the antigenic recombinant protein is at least 95%, 96%, 97%, 98%, 99%, or 100% identical to SEQ ID NO: 44 or 45.

3. The immunogenic composition of claim 1, wherein the antigenic recombinant protein is at least one selected from the group consisting of
an S-2P Omicron BA.1 recombinant protein comprising an amino acid sequence at least 98%, 99%, or 100% identical to SEQ ID NO: 14 or 15;
an S-2P Omicron BA.4/BA.5 recombinant protein comprising an amino acid sequence at least 98%, 99%, or 100% identical to SEQ ID NO: 17 or 18;
an S-2P Omicron BA.2.75 recombinant protein comprising an amino acid sequence at least-98%, 99%, or 100% identical to SEQ ID NO: 20 or 21:
an S-2P Omicron BA.2 recombinant protein comprising an amino acid sequence at least 98%, 99%, or 100% identical to SEQ ID NO: 22 or 23;
an S-2P Omicron BA.4.6 recombinant protein comprising an amino acid sequence at least 98%, 99%, or 100% identical to SEQ ID NO: 24 or 25;
an S-2P Omicron BF.7 recombinant protein comprising an amino acid sequence at least 98%, 99%, or 100% identical to SEQ ID NO: 26 or 27;
an S-2P Omicron BQ.1 recombinant protein comprising an amino acid sequence at least 98%, 99%, or 100% identical to SEQ ID NO: 28 or 29;
an S-2P Omicron BQ.1.1 recombinant protein comprising an amino acid sequence at least 98%, 99%, or 100% identical to SEQ ID NO: 30 or 31;
an S-2P Omicron XBB.1.5 recombinant protein comprising an amino acid sequence at least 98%, 99%, or 100% identical to SEQ ID NO: 32 or 33;
an S-2P Omicron XBB.1 recombinant protein comprising an amino acid sequence at least 98%, 99%, or 100% identical to SEQ ID NO: 34 or 35;
an S-2P Omicron XBB.1.16 recombinant protein comprising an amino acid sequence at least 98%, 99%, or 100% identical to SEQ ID NO: 36 or 37:
an S-2P Omicron BA.2.86 recombinant protein comprising an amino acid sequence at least 98%, 99%, or 100% identical to SEQ ID NO: 38 or 39; and
an S-2P Omicron EG.5.1 recombinant protein comprising an amino acid sequence at least 98%, 99%, or 100% identical to SEQ ID NO: 40 or 41.

4. The immunogenic composition of claim 1, further comprising an S-2P W recombinant protein, substantially consisting of residues 14-1208 of spike protein of SARS-CoV-2 Wuhan strain with proline substitutions at residues 986 and 987 and a “GSAS” substitution at residues 682-685 and a C-terminal T4 fibritin trimerization motif of SEQ ID NO: 2, with or without a cleaved HRV3C protease cleavage sequence of SEQ ID NO: 19 at a C-terminus.

5. The immunogenic composition of claim 4, wherein the S-2P W recombinant protein comprises an amino acid sequence at least 98%, 99%, or 100% identical to SEQ ID NO: 5 or 6.

6. The immunogenic composition of claim 1, wherein the aluminum-containing adjuvant comprises aluminum hydroxide, aluminum oxyhydroxide, aluminum hydroxide gel, aluminum phosphate, aluminum phosphate gel, aluminum hydroxyphosphate, aluminum hydroxyphosphate sulfate, amorphous aluminum hydroxyphosphate sulfate, potassium aluminum sulfate, aluminum monostearate or a combination thereof.

7. The immunogenic composition of claim 6, wherein a 0.5 ml dose of the immunogenic composition comprises from about 250 to about 1500 μg Al³⁺, or about 375 μg Al³⁺ or about 750 μg Al³⁺.

8. The immunogenic composition of claim 1, wherein the unmethylated CpG motif comprises a synthetic oligodeoxynucleotide (ODN) of SEQ ID NO: 7, SEQ ID NO: 8, SEQ ID NO: 9, SEQ ID NO: 10, SEQ ID NO: 11, SEQ ID NO: 12, or a combination thereof.

9. The immunogenic composition of claim 8, wherein a 0.5 ml dose of the immunogenic composition comprises from about 750 to about 3000 μg of the synthetic oligodeoxynucleotide, or about 750 μg, 1500 μg, or 3000 μg of the synthetic oligodeoxynucleotide.

10. A method for eliciting an immune response against a severe acute respiratory syndrome coronavirus 2 (SARS-CoV-2) in a subject in need thereof, comprising administering to the subject at least one dose of the immunogenic composition of claim 1.

11. The method of claim 10, wherein the subject was administered with at least one dose of an immunogenic composition derived from SARS-CoV-2 Wuhan strain before the administration with the at least one dose of the immunogenic composition of claim 1.

12. The method of claim 11, wherein the immunogenic composition derived from SARS-CoV-2 Wuhan strain comprises a polynucleotide encoding at least a fragment of spike protein of SARS-CoV-2 Wuhan strain or a polypeptide of at least a fragment of spike protein of SARS-CoV-2 Wuhan strain.

13. The method of claim 12, wherein the at least a fragment of spike protein of SARS-CoV-2 Wuhan strain comprises a polypeptide sequence at least 98%, 99%, or 100% identical to SEQ ID NO: 5 or 6.

14. The method of claim 11, wherein the immunogenic composition derived from SARS-CoV-2 Wuhan strain comprises a polypeptide sequence at least 98%, 99%, or 100% identical to SEQ ID NO: 5 or 6, and an adjuvant selected from the group consisting of an aluminum-containing adjuvant, an unmethylated cytosine-phosphate-guanosine (CpG) motif, and a combination thereof.

15. The method of claim 14, wherein the aluminum-containing adjuvant comprises aluminum hydroxide, aluminum oxyhydroxide, aluminum hydroxide gel, aluminum phosphate, aluminum phosphate gel, aluminum hydroxyphosphate, aluminum hydroxyphosphate sulfate, amorphous aluminum hydroxyphosphate sulfate, potassium aluminum sulfate, aluminum monostearate or a combination thereof.

16. The method of claim 15, wherein a 0.5 ml dose of the immunogenic composition comprises from about 250 to about 1500 μg Al³⁺, or about 375 μg Al⁺ or about 750 μg Al³⁺.

17. The method of claim 14, wherein the unmethylated CpG motif comprises a synthetic oligodeoxynucleotide (ODN) of SEQ ID NO: 7, SEQ ID NO: 8, SEQ ID NO: 9, SEQ ID NO: 10, SEQ ID NO: 11, SEQ ID NO: 12, or a combination thereof.

18. The method of claim 17, wherein a 0.5 ml dose of the immunogenic composition comprises from about 750 to about 3000 μg of the synthetic oligodeoxynucleotide, or about 750 μg, 1500 μg, or 3000 μg of the synthetic oligodeoxynucleotide.

19. The method of claim 11, wherein the severe acute respiratory syndrome coronavirus 2 (SARS-CoV-2) is an Omicron variant.

20. The method of claim 11, wherein the immunogenic composition is administered by intramuscular injection.

21. The immunogenic composition of claim 1 for use in eliciting an immune response against a severe acute respiratory syndrome coronavirus 2 (SARS-CoV-2) in a subject in need thereof.

22. The immunogenic composition of claim 1 for use according to claim 21, wherein the subject was administered with at least one dose of an immunogenic composition derived from SARS-CoV-2 Wuhan strain before the administration with at least one dose of the immunogenic composition of claim 1.

23. The immunogenic composition of claim 1 for use according to claim 22, wherein the immunogenic composition derived from SARS-CoV-2 Wuhan strain comprises a polynucleotide encoding at least a fragment of spike protein of SARS-CoV-2 Wuhan strain or a polypeptide of at least a fragment of spike protein of SARS-CoV-2 Wuhan strain.

24. The immunogenic composition of claim 1 for use according to claim 23, wherein the at least a fragment of spike protein of SARS-CoV-2 Wuhan strain comprises a polypeptide sequence at least 98%, 99%, or 100% identical to SEQ ID NO: 5 or 6.

25. The immunogenic composition of claim 1 for use according to claim 22, wherein the immunogenic composition derived from SARS-CoV-2 Wuhan strain comprises a polypeptide sequence at least 98%, 99%, or 100% identical to SEQ ID NO: 5 or 6, and an adjuvant selected from the group consisting of an aluminum-containing adjuvant, an unmethylated cytosine-phosphate-guanosine (CpG) motif, and a combination thereof.

26. The immunogenic composition of claim 1 for use according to claim 25, wherein the aluminum-containing adjuvant comprises aluminum hydroxide, aluminum oxyhydroxide, aluminum hydroxide gel, aluminum phosphate, aluminum phosphate gel, aluminum hydroxyphosphate, aluminum hydroxyphosphate sulfate, amorphous aluminum hydroxyphosphate sulfate, potassium aluminum sulfate, aluminum monostearate or a combination thereof.

27. The immunogenic composition of claim 1 for use according to claim 26, wherein a 0.5 ml dose of the immunogenic composition comprises from about 250 to about 1500 μg Al³⁺, or about 375 μg Al³⁺ or about 750 μg Al³⁺.

28. The immunogenic composition of claim 1 for use according to claim 25, wherein the unmethylated CpG motif comprises a synthetic oligodeoxynucleotide (ODN) of SEQ ID NO: 7, SEQ ID NO: 8, SEQ ID NO: 9, SEQ ID NO: 10, SEQ ID NO: 11, SEQ ID NO: 12, or a combination thereof.

29. The immunogenic composition of claim 1 for use according to claim 28, wherein a 0.5 ml dose of the immunogenic composition comprises from about 750 to about 3000 μg of the synthetic oligodeoxynucleotide, or about 750 μg, 1500 μg, or 3000 μg of the synthetic oligodeoxynucleotide.

30. The immunogenic composition of claim 1 for use according to claim 21, wherein the severe acute respiratory syndrome coronavirus 2 (SARS-CoV-2) is the Omicron variant.

31. The immunogenic composition of claim 1 for use according to claim 21, wherein the immunogenic composition is administered by intramuscular injection.

32. Use of the immunogenic composition of claim 1 for the manufacture of a medicament for eliciting an immune response against a severe acute respiratory syndrome coronavirus 2 (SARS-CoV-2) in a subject in need thereof.

33. The use according to claim 32, wherein the subject was administered with at least one dose of an immunogenic composition derived from SARS-CoV-2 Wuhan strain before the administration with at least one dose of the immunogenic composition of claim 1.

34. The use according to claim 33, wherein the immunogenic composition derived from SARS-CoV-2 Wuhan strain comprises a polynucleotide encoding at least a fragment of spike protein of SARS-CoV-2 Wuhan strain or a polypeptide of at least a fragment of spike protein of SARS-CoV-2 Wuhan strain.

35. The use according to claim 34, wherein the at least a fragment of spike protein of SARS-CoV-2 Wuhan strain comprises a polypeptide sequence at least 98%, 99%, or 100% identical to SEQ ID NO: 5 or 6.

36. The use according to claim 33, wherein the immunogenic composition derived from SARS-CoV-2 Wuhan strain comprises a polypeptide sequence at least 98%, 99%, or 100% identical to SEQ ID NO: 5 or 6, and an adjuvant selected from the group consisting of an aluminum-containing adjuvant, an unmethylated cytosine-phosphate-guanosine (CpG) motif, and a combination thereof.

37. The use according to claim 36, wherein the aluminum-containing adjuvant comprises aluminum hydroxide, aluminum oxyhydroxide, aluminum hydroxide gel, aluminum phosphate, aluminum phosphate gel, aluminum hydroxyphosphate, aluminum hydroxyphosphate sulfate, amorphous aluminum hydroxyphosphate sulfate, potassium aluminum sulfate, aluminum monostearate or a combination thereof.

38. The use according to claim 37, wherein a 0.5 ml dose of the immunogenic composition comprises from about 250 to about 1500 μg Al³⁺, or about 375 μg Al³⁺ or about 750 μg Al³⁺.

39. The use according to claim 36, wherein the unmethylated CpG motif comprises a synthetic oligodeoxynucleotide (ODN) of SEQ ID NO: 7, SEQ ID NO: 8, SEQ ID NO: 9, SEQ ID NO: 10, SEQ ID NO: 11, SEQ ID NO: 12, or a combination thereof.

40. The use according to claim 39, wherein a 0.5 ml dose of the immunogenic composition comprises from about 750 to about 3000 μg of the synthetic oligodeoxynucleotide, or about 750 μg, 1500 μg, or 3000 μg of the synthetic oligodeoxynucleotide.

41. The use according to claim 32, wherein the severe acute respiratory syndrome coronavirus 2 (SARS-CoV-2) is the Omicron variant.

42. The use according to claim 32, wherein the immunogenic composition is administered by intramuscular injection.


SEQUENCE LISTING (45 sequences)

SEQ ID NO: 1 - protein, 1208 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 2 - protein, 32 aa, unidentified
GSGYIPEAPRDGQAYVRKDGEWVLLSTFLGRS

SEQ ID NO: 3 - protein, 9 aa, unidentified
LEVLFQGPG

SEQ ID NO: 4 - protein, 31 aa, unidentified
SAWSHPQFEKGGGSGGGGSGGSAWSHPQFEK

SEQ ID NO: 5 - protein, 1227 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRS

SEQ ID NO: 6 - protein, 1233 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQ

SEQ ID NO: 7 - DNA, 22 bp, unidentified
  source 1..22: mol_type = other DNA
tgactgtgaacgttcgagatga

SEQ ID NO: 8 - DNA, 24 bp, unidentified
  source 1..24: mol_type = other DNA
tcgtcgttttgtcgttttgtcgtt

SEQ ID NO: 9 - DNA, 20 bp, unidentified
  source 1..20: mol_type = other DNA
ggtgcatcgatgcagggggg

SEQ ID NO: 10 - DNA, 22 bp, unidentified
  source 1..22: mol_type = other DNA
tccatggacgttcctgagcgtt

SEQ ID NO: 11 - DNA, 22 bp, unidentified
  source 1..22: mol_type = other DNA
tcgtcgttcgaacgacgttgat

SEQ ID NO: 12 - DNA, 23 bp, unidentified
  source 1..23: mol_type = other DNA
tcgtcgacgatcggcgcgcgccg

SEQ ID NO: 13 - protein, 1205 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 14 - protein, 1224 aa, synthetic construct
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRS

SEQ ID NO: 15 - protein, 1230 aa, synthetic construct
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQ

SEQ ID NO: 16 - protein, 1203 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 17 - protein, 1222 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRS

SEQ ID NO: 18 - protein, 1228 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQ

SEQ ID NO: 19 - protein, 6 aa, synthetic construct
LEVLFQ

SEQ ID NO: 20 - protein, 1224 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHENNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGFNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRS

SEQ ID NO: 21 - protein, 1230 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHENNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGFNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQ

SEQ ID NO: 22 - protein, 1224 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRS

SEQ ID NO: 23 - protein, 1230 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQ

SEQ ID NO: 24 - protein, 1222 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNSSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRS

SEQ ID NO: 25 - protein, 1228 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNSSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQ

SEQ ID NO: 26 - protein, 1222 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRS

SEQ ID NO: 27 - protein, 1228 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQ

SEQ ID NO: 28 - protein, 1222 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRS

SEQ ID NO: 29 - protein, 1228 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQ

SEQ ID NO: 30 - protein, 1222 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRS

SEQ ID NO: 31 - protein, 1228 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQ

SEQ ID NO: 32 - protein, 1223 aa, synthetic construct
QCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRS

SEQ ID NO: 33 - protein, 1229 aa, synthetic construct
QCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQ

SEQ ID NO: 34 - protein, 1223 aa, synthetic construct
QCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGSNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRS

SEQ ID NO: 35 - protein, 1229 aa, synthetic construct
QCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGSNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQ

SEQ ID NO: 36 - protein, 1223 aa, synthetic construct
QCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNRPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRS

SEQ ID NO: 37 - protein, 1229 aa, synthetic construct
QCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNRPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQ

SEQ ID NO: 38 - protein, 1219 aa, synthetic construct
QCVNLITTTQSYTNSFTRGVYYPDKVFRSSVLHLTQDLFLPFFSNVTWFHAISGTNGTKRFDNPVLPFNDGVYFASTEKSNIIRGWIFGTTLDSKTQSLLIVNNATNVFIKVCEFQFCNDPFLDVYHKNNKSWMESESGVYSSANNCTFEYVSQPFLMDLEGKQGNFKNLREFVFKNIDGYFKIYSKHTPIIGRDFPQGFSALEPLVDLPIGINITRFQTLLALNRSYLTPGDSSSGWTAGAADYYVGYLQPRTFLLKYNENGTITDAVDCALDPLSETKCTLKSFTVEKGIYQTSNFRVQPTESIVRFPNVTNLCPFHEVFNATRFASVYAWNRTRISNCVADYSVLYNFAPFFAFKCYGVSPTKLNDLCFTNVYADSFVIKGNEVSQIAPGQTGNIADYNYKLPDDFTGCVIAWNSNKLDSKHSGNYDYWYRLFRKSKLKPFERDISTEIYQAGNKPCKGKGPNCYFPLQSYGFRPTYGVGHQPYRVVVLSFELLHAPATVCGPKKSTNLVKNKCVNFNFNGLTGTGVLTKSNKKFLPFQQFGRDIVDTTDAVRDPQTLEILDITPCSFGGVSVITPGTNTSNQVAVLYQGVNCTEVSVAIHADQLTPTWRVYSTGSNVFQTRAGCLIGAEYVNNSYECDIPIGAGVCASYQTQTKSRGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLFSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQLELDSFKEELDKYFKNHTSPDVDLGDISGINASVVNIQKEIDRLNEVAKNLNESLIDLQELGKYEQGSGYIPEAPRDGQAYVRKDGEWVLLSTFLGRS

SEQ ID NO: 39 - protein, 1225 aa, synthetic construct
QCVNLITTTQSYTNSFTRGVYYPDKVFRSSVLHLTQDLFLPFFSNVTWFHAISGTNGTKRFDNPVLPFNDGVYFASTEKSNIIRGWIFGTTLDSKTQSLLIVNNATNVFIKVCEFQFCNDPFLDVYHKNNKSWMESESGVYSSANNCTFEYVSQPFLMDLEGKQGNFKNLREFVFKNIDGYFKIYSKHTPIIGRDFPQGFSALEPLVDLPIGINITRFQTLLALNRSYLTPGDSSSGWTAGAADYYVGYLQPRTFLLKYNENGTITDAVDCALDPLSETKCTLKSFTVEKGIYQTSNFRVQPTESIVRFPNVTNLCPFHEVFNATRFASVYAWNRTRISNCVADYSVLYNFAPFFAFKCYGVSPTKLNDLCFTNVYADSFVIKGNEVSQIAPGQTGNIADYNYKLPDDFTGCVIAWNSNKLDSKHSGNYDYWYRLFRKSKLKPFERDISTEIYQAGNKPCKGKGPNCYFPLQSYGFRPTYGVGHQPYRVVVLSFELLHAPATVCGPKKSTNLVKNKCVNFNFNGLTGTGVLTKSNKKFLPFQQFGRDIVDTTDAVRDPQTLEILDITPCSFGGVSVITPGTNTSNQVAVLYQGVNCTEVSVAIHADQLTPTWRVYSTGSNVFQTRAGCLIGAEYVNNSYECDIPIGAGVCASYQTQTKSRGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLFSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQLELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQ

SEQ ID NO: 40 - protein, 1223 aa, synthetic construct
QCVNLITRTQSYTNSFTRGVYYPDKVFRSSVLHSTHDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLLRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRS

SEQ ID NO: 41 - protein, 1229 aa, synthetic construct
QCVNLITRTQSYTNSFTRGVYYPDKVFRSSVLHSTHDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLLRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQ

SEQ ID NO: 42 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 43 - protein, 1273 aa, unidentified
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSAPFFTFKCYGVSPTKLNDLCFTNVYADSFVIRGDEVRQIAPGQTGNIADYNYKLPDDFTGCVIAWNSNKLDSKVGGNYNYLYRLFRKSNLKPFERDISTEIYQAGNKPCNGVAGFNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 44 - protein, 1227 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSAPFFTFKCYGVSPTKLNDLCFTNVYADSFVIRGDEVRQIAPGQTGNIADYNYKLPDDFTGCVIAWNSNKLDSKVGGNYNYLYRLFRKSNLKPFERDISTEIYQAGNKPCNGVAGFNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRS

SEQ ID NO: 45 - protein, 1233 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSAPFFTFKCYGVSPTKLNDLCFTNVYADSFVIRGDEVRQIAPGQTGNIADYNYKLPDDFTGCVIAWNSNKLDSKVGGNYNYLYRLFRKSNLKPFERDISTEIYQAGNKPCNGVAGFNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQ